(19) United States
(12) Patent Application Publication
(10) Pub. No.: US 2024/0399160 A1
(43) Pub. Date: Dec. 5, 2024

(54) IMPLANTABLE MEDICAL DEVICE FOR PROVIDING A DIAGNOSTIC AND/OR THERAPEUTIC CARDIAC FUNCTION WITHIN A PATIENT

(71) Applicant: BIOTRONIK SE & Co. KG, Berlin (DE)

(72) Inventors: Thomas DOERR, Berlin (DE); Ingo WEISS, Berlin (DE)

(73) Assignee: BIOTRONIK SE & Co. KG, Berlin (DE)

(21) Appl. No.: 18/713,930

(22) PCT Filed: Nov. 28, 2022
(86) PCT No.: PCT/EP2022/083433

§ 371 Date: May 28, 2024

(30) Foreign Application Priority Data
Dec. 1, 2021 (EP) 21211619.8

(51) Int. Cl. A61N 1/39; A61N 1/365; A61N 1/375

(52) U.S. Cl. CPC A61N 1/3925; A61N 1/365; A61N 1/3756; A61N 1/39622

(57) ABSTRACT

An implantable medical device for providing a diagnostic and/or therapeutic cardiac function within a patient comprises a sensing arrangement for sensing electrocardiogram signals, a communication circuitry for data communication with a device external to the patient and a processing circuitry for processing sensed electrocardiogram signals. The processing circuitry is configured to identify, based on the sensed electrocardiogram signals, a stimulation event caused by an implantable pacemaker device and to cause a transmission of information concerning said identification of a stimulation event to the device external to the patient.

### CROSS-REFERENCE TO RELATED APPLICATIONS

This application is the United States National Phase under 35 U.S.C. § 371 of PCT International Patent Application No. PCT/EP2022/083433, filed on Nov. 28, 2022, which claims the benefit of European Patent Application No. 21211619.8, filed on Dec. 1, 2021, the disclosures of which are hereby incorporated by reference herein in their entireties.

### TECHNICAL FIELD

The instant invention generally relates to an implantable medical device for providing a diagnostic and/or therapeutic cardiac function within a patient, to an implantable therapy system and to a method for operating an implantable medical device for providing a diagnostic and/or therapeutic cardiac function within a patient.

### BACKGROUND

Generally, an implantable medical device comprises a sensing arrangement for sensing electrocardiogram signals, a communication circuitry for data communication with a device external to the patient, and a processing circuitry for processing sensed electrocardiogram signals.

According to an embodiment of the present invention, the communication circuitry for data communication is configured to communicate with a device external to the patient or with an external server.

An implantable medical device of the type concerned herein, for example, may be an implantable cardioverter defibrillator (ICD), which in particular may be configured for a non-transvenous implantation external to the patient's heart.

Within an implantable therapy system, multiple implantable medical devices may be implanted in a patient, for example an implantable medical device in the shape of an implantable cardioverter defibrillator together with a pacemaker device, such as a leadless pacemaker. Devices of the implantable therapy system herein shall communicate with an external device, for example within a home monitoring system, such that information concerning devices of the implantable therapy system is communicated to the external device and, via the external device, may be relayed, e.g., to a home monitoring service center for assessment, e.g., by a physician.

Within an implantable therapy system, some devices may be implanted deeper in a patient than others. In addition, some devices, such as, for example, a leadless pacemaker device, may be small in construction and hence may comprise only a comparatively small battery comprising a limited energy storage capacity, such that those devices shall operate in a particularly energy-efficient manner.

In order to allow for a communication between devices of an implantable therapy system, approaches exist for establishing an intra-body communication network. Intra-body communication techniques however require the implantable medical devices to interact in a defined manner, posing rather strict compatibility restrictions on the implantable medical devices.

In other approaches, a communication with implantable medical devices implanted deep in a patient may involve low frequency communication techniques, for example employing a coil telemetry communication. This however requires the use of a reading device which must be approached externally to the patient in order to establish a data transmission, making a communication potentially unreliable and cumbersome for a user.

European Patent No. 2 327 609 B1 describes an acoustic communication link in between implanted medical devices for exchanging information in between the implanted medical devices. The acoustic communication link is established to permit wireless communication between the implanted medical devices, wherein transmission parameters may be adapted, such as a sensitivity and a carrier frequency, in order to improve an existing communication link.

U.S. Publication No. 2010/0022836 A1 discloses multidirectional transmitters for in-body devices, such as implantable devices.

The present disclosure is directed toward overcoming one or more of the above-mentioned problems, though not necessarily limited to embodiments that do.

### SUMMARY

It is an object of the instant invention to provide an implantable medical device, an implantable therapy system and a method for operating an implantable medical device which allow for an easy and reliable monitoring and information transmission concerning a device which may be implanted deep in a patient.

At least this object is achieved by means of an implantable medical device comprising the features of claim 1.

In one aspect, an implantable medical device for providing a diagnostic and/or therapeutic cardiac function within a patient, comprises: a sensing arrangement for sensing electrocardiogram signals: a communication circuitry for data communication with a device external to the patient; and a processing circuitry for processing sensed electrocardiogram signals, wherein the processing circuitry is configured to identify, based on the sensed electrocardiogram signals, a stimulation event caused by an implantable pacemaker device and to cause a transmission of information concerning said identification of a stimulation event to the device external to the patient.

The implantable medical device may in particular be an implantable non-transvenous defibrillator device configured to emit a shock pulse for achieving a defibrillation. An implantable non-transvenous defibrillator device, also denoted as non-transvenous implantable cardioverter defibrillator (in short non-transvenous ICD), may serve for monitoring and treating potentially life-threatening arrhythmias of a patient's heart. The implantable non-transvenous defibrillator device is configured for non-transvenous implantation, that is an implantation such that no electrode leads transvenously are implanted within the heart of a (human or animal) patient. The implantable non-transvenous defibrillator device hence is to be implanted in a patient such that a generator and an electrode arrangement, for example a shock electrode placed on a lead connected to the generator, are implanted extracardially and do not reach into the heart of the patient, that is into the right or left ventricle or the right or left atrium.

In one embodiment, the implantable non-transvenous defibrillator device comprises a lead to be implanted extracardially and a shock electrode arranged on the lead. The shock electrode herein, in an implanted state of the defibrillator device, is placed outside of the heart of the patient, for example in the region of the sternum of the patient, such that a shock pulse for achieving a defibrillation is generated outside of the heart. Generally, the implantable non-transvenous defibrillator device does not comprise any portions which extend transvenously into the heart, but the defibrillator device is configured to achieve a sensing and emission of signals outside of the heart.

The implantable medical device comprises a sensing arrangement for sensing electrocardiogram signals. The sensing arrangement may, for example, comprise one or multiple pairs of electrode poles via which electrocardiogram signals may be sensed and forwarded to processing circuitry operatively connected to the sensing arrangement.

The processing circuitry is configured to process sensed electrocardiogram signals, wherein the processing circuitry is configured to identify, within the sensed electrocardiogram signals, stimulation events caused by an implantable pacemaker device. The implantable medical device hence is programmed to derive, from sensed electrocardiogram signals, information relating to another implantable device, namely an implantable pacemaker device, which is separate and independent from the implantable medical device. The implantable pacemaker device in particular may be a leadless pacemaker device which is directly implanted into the heart of the patient, for example into the right or left ventricle, and which does not comprise leads carrying electrodes.

In that the implantable medical device, which may in particular be implanted extracardially, identifies stimulation events as caused by the implantable pacemaker device within sensed electrocardiogram signals and communicates information concerning the identification of stimulation events to an external device, the implantable medical device may establish a monitoring function for the implantable pacemaker device. Information relating to the operation of the implantable medical device hence may be monitored by the implantable medical device and may be communicated by the implantable medical device to an external device. In this way data concerning the operation of the implantable pacemaker device is obtained using the implantable medical device and is communicated to the external device by means of the implantable medical device, hence making a direct data communication in between the implantable pacemaker device and the external device dispensable.

In principle, hence, the implantable pacemaker device does not need to establish a communication to the external device, such that the implantable pacemaker device may be operated in a particularly energy-efficient manner.

A monitoring of the operation of the implantable pacemaker device herein may reliably be established even if the implantable pacemaker device is implanted deep in the patient, hence making a direct data communication of the implantable pacemaker device to an external device potentially difficult. In that the monitoring of the implantable pacemaker device is carried out by the implantable medical device based on the sensing of electrocardiogram signals, no data transmission from the implantable pacemaker device to an external device is required to establish a monitoring, such that a deep implantation state of the implantable pacemaker device does not impact data communication.

The implantable medical device is configured to identify stimulation events caused by the implantable pacemaker device based on sensed electrocardiogram signals. Within regular operation the implantable medical device records electrocardiogram signals and processes sensed electrocardiogram signals, wherein based on the processing stimulation events as caused by the implantable pacemaker device are identified and information relating to the stimulation events is derived and is transmitted to the external device. As the identification of the stimulation events does not require a direct communication in between the implantable pacemaker device and the implantable medical device, requirements for a compatibility of the devices are negligible. A monitoring of an implantable pacemaker device hence may be established, for example, even in scenarios in which the implantable pacemaker device and the implantable medical device originate from different manufacturers.

In one embodiment, the processing circuitry is configured to store a portion of the sensed electrocardiogram signals dependent on said identification of a stimulation event and to transmit a stored portion of the sensed electrocardiogram signals to the device external to the patient. If a stimulation event is identified, signal portions relating to the stimulation event may be stored and may be communicated to the external device, such that signal portions indicative of the stimulation events are forwarded to the external device for further processing.

In one embodiment, the processing circuitry comprises a circular buffer for circularly storing sensed electrocardiogram signals. The circular buffer, also denoted as ring memory, may, for example, comprise a storage capacity for storing a signal portion of a specified time length, for example in a range between 10 seconds and 5 minutes, in particular in between seconds and 1 minute. The circular buffer herein overwrites circularly its memory 20 contents, such that the circular buffer at any time contains a signal portion of a defined prior time span.

In case a stimulation event caused by the implantable pacemaker device is identified, the processing circuitry may cause a transmission of a data message containing a signal portion prior to the stimulation event, a signal portion containing the stimulation event and/or a signal portion after the stimulation event. Resulting from the identification of a stimulation event, hence, a signal portion is extracted from the circular buffer and is forwarded to the external device, such that electrocardiogram signals indicative of the stimulation event are transmitted to the external device.

Alternatively or in addition, the processing circuitry may be configured to statistically analyze stimulation events as identified based on the electrocardiogram signals. Statistical information concerning identified stimulation events may then be derived by the processing circuitry and may be communicated to the external device.

In one embodiment, the processing circuitry is configured to store programming information indicative of a programmed stimulation function of the implantable pacemaker device, wherein the processing circuitry is configured to monitor the programmed stimulation function based on the identification of a stimulation event. The implantable medical device hence may comprise information relating to the operation of the implantable pacemaker device. The processing circuitry of the implantable medical device is programmed such that it is enabled to assess how the implantable pacemaker device should function and to evaluate whether the implantable pacemaker device functions correctly according to its programmed stimulation function. If it is found that the implantable pacemaker device in its operation deviates from the programmed stimulation function, information in this respect, including, for example, a warning message, may be sent to the external device.

For example, the processing circuitry may be configured to identify, based on the sensed electrocardiogram signals, a requirement for a stimulation event and to monitor, based on the sensed electrocardiogram signals, whether the implantable pacemaker device causes a stimulation event in relation to an identification of a requirement for a stimulation event. If the processing circuitry identifies, based on the sensed electrocardiogram signals, that in a particular situation the implantable pacemaker device should trigger a stimulation event based on a programmed stimulation function of the implantable pacemaker device, the processing circuitry monitors the sensed electrocardiogram signals whether the stimulation event in fact occurs. If the stimulation event occurs, the processing circuitry of the implantable medical device may identify that the implantable pacemaker device functions according to its prescribed programming. If the stimulation event does not occur, the processing circuitry may identify that the implantable pacemaker device does not operate according to its programmed stimulation function and may accordingly send information to the external device.

In an example, the processing circuitry may be configured to identify an anti-tachycardia stimulation event caused by the implantable pacemaker device and to cause a transmission of information concerning the anti-tachycardia stimulation event to the external device. The programmed stimulation function of the implantable pacemaker device may, for example, relate to an anti-tachycardia stimulation (ATP) in order to provide a therapy for a ventricular tachycardia. If the implantable medical device identifies an anti-tachycardia stimulation event, information relating to the anti-tachycardia stimulation event may be communicated to the external device.

In another example, the processing circuitry may be configured to identify a requirement for an anti-bradycardia stimulation and to monitor whether the implantable pacemaker device causes an anti-bradycardia stimulation event in relation to an identification of a requirement for an anti-bradycardia stimulation. The implantable pacemaker device hence is programmed to provide for a therapy in case a bradycardia occurs. If the implantable medical device identifies a bradycardia and hence assesses that an anti-bradycardia stimulation event should be caused by the implantable pacemaker device, but in the further monitoring detects that no stimulation by the implantable pacemaker device takes place, this may indicate an incorrect functioning of the implantable pacemaker device, such that information in this respect, for example including a warning message, may be transmitted to the external device.

The processing circuitry of the implantable medical device generally may be configured to identify a stimulation event caused by the implantable pacemaker device and to monitor a subsequent stimulated cardiac response, for example in a predefined time window following the identified stimulation event. In this way the processing circuitry may monitor a cardiac response and may classify a stimulation, based on the cardiac response, as effective or ineffective. Information in this respect may be transmitted to the external device.

In one embodiment, the processing circuitry is configured to trigger a data communication message to the device external to the patient based on the identification of a stimulation event. Hence, in an event-based approach a data communication may be initiated, for example, immediately upon identifying a stimulation event or based on a monitoring event indicating a correct or incorrect stimulation operation by the implantable pacemaker device.

Alternatively or in addition, information concerning said identification of a stimulation event may be included in a data message which is transmitted by the implantable medical device according to a predefined, regular communication scheme. The implantable medical device may, for example, be configured to regularly send data messages to the external device. In such regular messages also information concerning the implantable pacemaker device may be included.

In an embodiment of the present invention, the implantable pacemaker device is configured to deliver at least one specific stimulation pattern to indicate at least one device condition. The processing circuitry of the implantable medical device is configured to sense and detect the specific stimulation pattern and forwards the information on the device state to the device external to the patient or to an external server. According to an embodiment, the specific pulse pattern comprises a pulse sequence which is unconventional for therapeutic cardiac pacing, as for instance a predetermined number of pulses at 70 bpm. In an embodiment, the specific pulse pattern comprises a repetition of the pulse sequence a predetermined number of times, e.g., 3 or 5 times.

According to an embodiment of the present invention, the device state may indicate an ERI (Elective replacement indication) status, calling for a battery change of the implantable pacemaker. In general, the status can be any other status which is crucial to report to an external user in order to trigger an external action.

In an embodiment of the present invention, the processing circuitry of the implantable medical device is configured to determine a mean stimulation rate of successive stimulation events caused by the implantable pacemaker device. In one example, the processing circuitry is configured to determine the mean stimulation rate by calculating a moving average. An example time window for ECG measurements for determining a moving average could be, e.g., 10-50s, in particular 30s.

Moreover, according to an embodiment, the processing circuitry is configured to track changes of the mean stimulation rate. For instance, in case the processing circuitry identifies a reduction of mean stimulation rate by 10% or 12% of the original mean stimulation rate.

A reduction of the stimulation rate can be automatically performed by the implantable pacemaker device when a low battery state is entered (e.g., ERI state), in order to save energy. By the said embodiment, the implantable medical device is enabled to detect that state change of the implantable pacemaker device, and forward the information of the state change to the device external to the patient or to an external server.

An implantable therapy system comprises, in one embodiment, an implantable pacemaker device for emitting a cardiac stimulation signal for achieving an intracardiac pacing, and an implantable medical device of the type described above. The implantable pacemaker device may, for example, be a leadless pacemaker device which is to be implanted directly into the heart, for example the right or left ventricle or the right or left atrium. The pacemaker device may, for example, provide for a therapy in case a bradycardia is detected in order to pace the patient's heart and to in this way pace the heart rate to a desired, normal heart rate. Alternatively or in addition, the pacemaker device may provide for a therapy in case a tachycardia is detected in order to reset the heart rate to a desired normal rate.

In another aspect, a method for operating an implantable medical device for providing a diagnostic and/or therapeutic cardiac function within a patient comprises: sensing, using a sensing arrangement of the implantable medical device, electrocardiogram signals; processing, using a processing circuitry of the implantable medical device, sensed electrocardiogram signals, wherein the processing includes: identifying, based on the sensed electrocardiogram signals, a stimulation event caused by an implantable pacemaker device and causing a transmission, using a communication circuitry of the implantable medical device, of information concerning an identification of a stimulation event to a device external to the patient.

The advantages and advantageous embodiments described above for the implantable medical device and the implantable therapy system equally apply also to the method, such that it shall be referred to the above in this respect.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The idea of the present invention shall subsequently be described in more detail with reference to the embodiments as shown in the drawings. Herein:

FIG. 1 shows a schematic drawing of a therapy system of medical devices implanted in a patient; and

FIG. 2 shows a schematic drawing of an implantable medical device together with an implantable pacemaker device.

### DETAILED DESCRIPTION

Subsequently, embodiments of the present invention shall be described in detail with reference to the drawings. In the drawings, like reference numerals designate like structural elements.

It is to be noted that the embodiments are not limiting for the present invention, but merely represent illustrative examples.

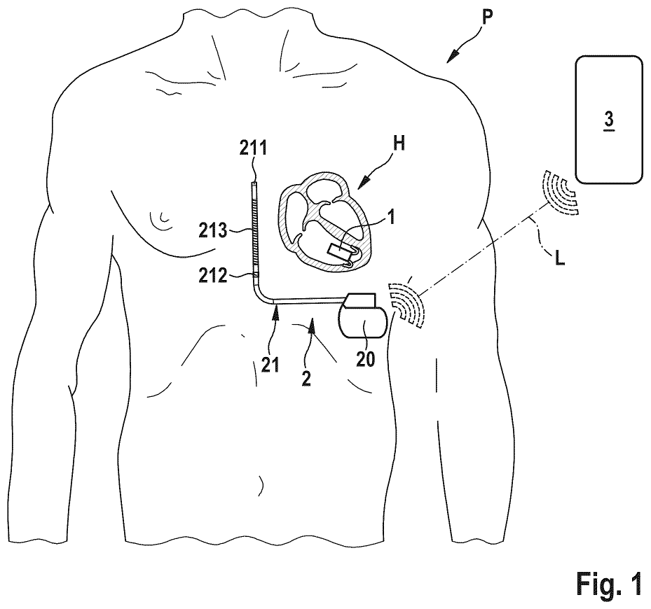

Referring to FIG. 1, in a setup of a therapy system an implantable leadless pacemaker device 1 is implanted in the heart H of a (human or animal) patient P. The leadless pacemaker device 1 is implanted directly into the heart H, for example in the right ventricle RV, in order to couple to tissue within the heart H and to in this way sense cardiac signals within the heart and emit stimulation signals into cardiac tissue.

In addition, an implantable medical device 2 in the shape of a non-transvenous implantable defibrillator device is implanted such that the implantable medical device 2 is completely external to the heart H, the non-transvenous implantable medical device 2 comprising a generator 20 encapsulated within a housing, and a lead 21 connected to the generator 20 and carrying sensing electrodes 211, 212 as well as a shock electrode 213 in the shape of a coil formed on a distal portion of the lead 21. The sensing electrodes 211, 212, for example formed as ring electrodes on either side of the shock electrode 213, serve to sense cardiac signals for processing within the generator 20 of the implantable medical device 2, such that based on sensed signals, e.g., a tachycardia may be identified and a shock pulse may be generated for providing for an anti-tachycardia therapy.

In a setup as shown in FIG. 1, the pacemaker device 1 implanted within the heart H and the implantable medical device 2 implanted outside of the heart H each comprise sensing circuitry, such that based on a processing of sensed signals in the respective device 1, 2 a therapeutic action may be initiated. Each device 1, 2 herein functions, in principle, independent of the other device 2, 1, such that by means of the pacemaker device 1 implanted in the heart H stimulation signals, e.g., for overcoming a bradycardia may be generated, whereas by means of the defibrillator device 2 a shock pulse may be emitted for resetting, e.g., a tachycardie heart rate to a normal heart rate.

In a setup as shown in FIG. 1, the implantable pacemaker device 1 is implanted in the heart H and hence rather deep within the patient P. The implantable medical device 2, in contrast, with its generator 20 may, for example, be implanted subcutaneously, the lead 21 extending from the generator 20 such that it extends outside of the heart H and hence does not reach transvenously into the heart H. The implantable medical device 2 is configured to establish a communication link L to an external device 3 such that a data communication in between the implantable medical device 2 and the external device 3 may take place.

The external device 3 may, for example, be part of a home monitoring system. The external device 3, for example, resides at the site, e.g., at the home, of the patient P and beneficially is in connection with a home monitoring service center, such that data may be relayed to and from the home monitoring center by means of the external device 3.

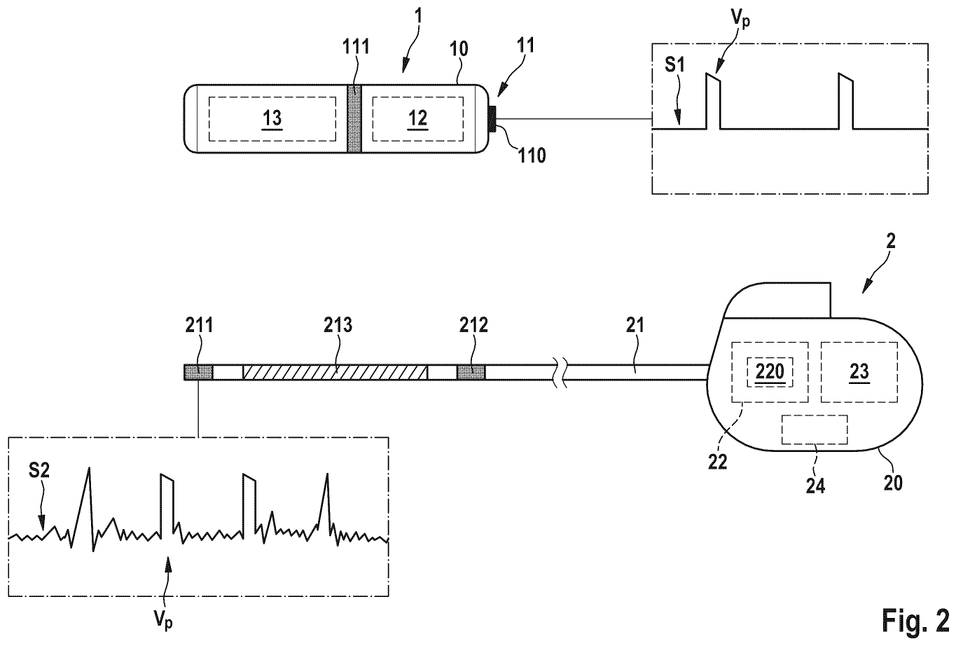

Referring now to FIG. 2, the pacemaker device 1 comprises a housing 10 on which an electrode arrangement 11 having electrode poles 110, 111 is arranged. The electrode poles 110, 111 form a pair of electrodes by means of which stimulation pulses may be injected into tissue and sense signals may be received from tissue to provide for a stimulation and sensing at the implantation site of the implantable pacemaker device 1. The implantable pacemaker device 1 furthermore comprises control circuitry 12 and a battery 13 providing for an energy storage, both the control circuitry 12 and the battery 13 being encapsulated in the housing 10.

The non-transvenous implantable medical device 2, within the generator 20, comprises processing circuitry 22, a battery 23 providing for an energy storage, and communication circuitry 24.

The processing circuitry 22 serves to process signals received via the sensing arrangement of the sensing electrodes 211, 212 and to trigger a stimulation action by emitting a shock pulse via the shock electrode 213 in case, e.g., a tachycardia is detected.

By means of the communication circuitry 24 a communication link L (see FIG. 1) to the external device 3 is established, the communication circuitry 24 being configured, for example, to establish a communication according to the MICS protocol, the ISM protocol, the BLE protocol, the Zigbee protocol, or according to another communication scheme.

The pacemaker device 1 and the non-transvenous implantable medical device 2, in the shown embodiment, are not functioning in immediate connection with each other. That is, the pacemaker device 1 and the implantable medical device 2 are not in data connection with each other, such that no control signals are exchanged in between the pacemaker device 1 and the implantable medical device 2.

Rather, the pacemaker device 1 functions, for example, without knowledge about the presence of the implantable medical device 2 by sensing electrocardiogram signals within the heart H and by emitting stimulation signals S1 based on a processing of sensed signals. The pacemaker device 1, for example, may be configured to detect a bradycardia, and to emit stimulation pulses to cause stimulation events Vp in case a bradycardia is detected.

In the example of FIGS. 1 and 2, the pacemaker device 1 does not directly communicate with the external device 3. In order to nevertheless provide for a monitoring of the operation of the pacemaker device 1, the implantable medical device 2 is configured to identify, within sensed electrocardiogram signals S2, stimulation events Vp caused by the pacemaker device 1.

For example, the sensed electrocardiogram signals S2 may be processed within the processing circuitry 22 of the generator 20 of the implantable medical device 2 by applying, e.g., a digitalization, an amplification and a filtering in order to discern a stimulation pulse of the implantable pacemaker device 1 from intrinsic electrocardiogram signals originating from cardiac activity. For example, the processing circuitry 22 may process sensed electrocardiogram signals S2 by assessing a positive or negative flank of a pulse, for example by evaluating the flank's gradient or the flank' height, or by applying pattern recognition techniques in order to identify a predefined pulse shape stemming from a stimulation pulse as emitted by the pacemaker device 1.

In one embodiment, the processing circuitry 22 comprises a circular buffer 220 which serves to circularly store sensed electrocardiogram signals S2 such that a portion of the sensed electrocardiogram signals S2 relating to a prior time span is always kept in memory and is circularly overwritten by new data. For example, the circular buffer 220 may be configured to keep a signal portion for a time span between 10 seconds and 5 minutes, for example between 20 seconds and 1 minute, in memory such that always a most recent signal portion is stored within the circular buffer 220.

The processing circuitry 22 is configured to derive information from the sensed electrocardiogram signals S2 in relation to the operation of the pacemaker device 1. The processing circuitry 22 hence is operative to enable a monitoring of the operation of the pacemaker device 1 based solely on the reception of electrocardiogram signals S2 and by assessing the action of the pacemaker device 1 according to sensed electrocardiogram signals S2. Based on the monitoring of the action of the pacemaker device 1, data may be transmitted to the external device 3 in order to provide for a monitoring of the operation of the pacemaker device 1.

Namely, in one embodiment the processing circuitry 22 is configured to cause a transmission of information to the external device 3 based on the identification of one or multiple stimulation events Vp within the sensed electrocardiogram signal S2. For example, in case a stimulation event Vp caused by the pacemaker device 1 is identified within the electrocardiogram signal S2, a signal portion containing the stimulation event Vp may be transmitted within a data message to the external device 3, such that the external device 3 obtains information about the stimulation event Vp as caused by the pacemaker device 1.

In another example, the processing circuitry 22 may derive statistical information based on stimulation events Vp and may transmit the statistical information to the external device 3. Statistical information may include a frequency of stimulation, an average period between stimulation events, information concerning a ratio of intrinsic versus stimulated activity, statistical information relating to a cardiac response, and other statistical data.

In yet another example, the processing circuitry 22 may have information about a programmed function of the pacemaker device 1 stored in its memory such that the implantable medical device 2 comprises information about an operative setup of the pacemaker device 1. The implantable medical device 2 hence is enabled to assess the operation of the pacemaker device 1, based on sensed electrocardiogram signals S2, versus an expected operation. If the pacemaker device 1 functions according to its expected operation, this may be protocolled. Likewise, if the operation of the pacemaker device 1 deviates from its expected functioning, this may be monitored and, for example, warning messages may be triggered.

For example, the pacemaker device 1 may be configured to provide for an anti-tachycardia stimulation in case the implantable pacemaker device 1 detects a tachycardia based on its sensed signals as received via the electrode arrangement 11. In case a tachycardia is detected, anti-tachycardia stimulation pulses shall be emitted. This operation by the pacemaker device 1 may be monitored by the implantable medical device 2, which in its sensed electrocardiogram signals S2 may likewise identify a tachycardia and, subsequent to the identification of a tachycardia, may monitor whether the pacemaker device 1 correctly emits an anti-tachycardia stimulation signal. Signal portions relating to the operation of the pacemaker device 1 may be stored, in case the pacemaker device 1 correctly emits an anti-tachycardia stimulation signal as well as in case the pacemaker device 1 does not emit an anti-tachycardia stimulation signal contrary to its expected behavior.

In another example, the pacemaker device 1 may be configured to provide for an anti-bradycardia stimulation in case a bradycardia is detected by the pacemaker device 1. The operation of the pacemaker device 1 is monitored by the implantable medical device 2 by identifying a bradycardia in the sensed electrocardiogram signal S2 and by, subsequently, monitoring whether the pacemaker device 1 correctly emits an anti-bradycardia pacing in accordance with the detected bradycardia.

Based on a monitoring of stimulation events Vp in the sensed electrocardiogram signals S2 as caused by the pacemaker device 1, the implantable medical device 2 may cause a data communication with the external device 3.

In an event-based communication, for example, the implantable medical device 2 may, for example, trigger a communication message on the event of detecting a stimulation event Vp as caused by the pacemaker device 1 or on the event of detecting a deviation within the operation of the pacemaker device 1 from an expected operation. Based on the detection of a certain event, hence, a data message is created and transmitted to the external device 3 using the communication circuitry 24.

In addition or alternatively, information concerning the pacemaker device 1 may be included in data messages as transmitted to the external device 3 according to a regular, pre-defined communication scheme, within which, for example, data messages are exchanged in between the implantable medical device 2 and the external device 3 in a regular, prescheduled manner. The identification of a stimulation event Vp or the identification of a deviation from an expected operation of the pacemaker device 1 thus does not cause a triggering of a specific, dedicated communication message, but information relating to the operation of the pacemaker device 1 is included in a regular communication message, for example to provide for a periodic reporting.

The data transmission may contain portions of the sensed electrocardiogram signals S2 relating to, e.g., an identified stimulation event Vp. In addition or alternatively, the data transmission may include information derived in connection with a stimulation event Vp, for example statistical information or timing information.

The idea of the present invention is not limited to the embodiments described above, but may be implemented in an entirely different fashion.

The implantable medical device may in particular be an implantable cardioverter defibrillator device (ICD). In another embodiment, the implantable medical device may be a monitoring device, such as a so-called biomonitor, to be implanted, e.g., subcutaneously in proximity to the heart for sensing and monitoring signals, which however does not by itself provide for a therapeutic function by emitting stimulation signals.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### LIST OF REFERENCE NUMERALS

1 Pacemaker device
10 Housing
11 Electrode arrangement
110, 111 Electrode poles
12 Control circuitry
13 Energy storage (battery)
2 Non-transvenous defibrillator device
20 Generator
21 Lead
211, 212 Sensing electrode
213 Shock electrode
22 Processing circuitry
220 Circular buffer
23 Energy storage (battery)
24 Communication circuitry
3 External device
H Heart
L Communication link
P Patient
S1 Stimulation signal
S2 Sensed signal
Vp Stimulation event

1. An implantable medical device for providing a diagnostic and/or therapeutic cardiac function within a patient, comprising:
a sensing arrangement for sensing electrocardiogram signals;
a communication circuitry for data communication with a device external to the patient; and
a processing circuitry for processing sensed electrocardiogram signals, wherein the processing circuitry is configured to identify, based on the sensed electrocardiogram signals, a stimulation event caused by an implantable pacemaker device and to cause a transmission of information concerning said identification of a stimulation event to the device external to the patient.

2. The implantable medical device according to claim 1, wherein the implantable medical device is an implantable non-transvenous defibrillator device configured to emit a shock pulse for achieving a defibrillation.

3. The implantable medical device according to claim 2, wherein the implantable non-transvenous defibrillator device comprises a lead to be implanted extracardially and a shock electrode arranged on the lead.

4. The implantable medical device according to claim 1, wherein the processing circuitry is configured to store a portion of the sensed electrocardiogram signals dependent on said identification of a stimulation event and to transmit a stored portion of the sensed electrocardiogram signals to the device external to the patient.

5. The implantable medical device according to claim 4, wherein the processing circuitry comprises a circular buffer for circularly storing the sensed electrocardiogram signals.

6. The implantable medical device according to claim 4, wherein said portion comprises a signal portion prior to the stimulation event, a signal portion containing the stimulation event and/or a signal portion after the stimulation event.

7. The implantable medical device according to claim 1, wherein the processing circuitry configured to statistically analyze a multiplicity of stimulation events caused by the implantable pacemaker device and to communicate statistical information concerning said multiplicity of stimulation events to the device external to the patient.

8. The implantable medical device according to claim 1, wherein the processing circuitry is configured to store programming information indicative of a programmed stimulation function of the implantable pacemaker device, wherein the processing circuitry is configured to monitor said programmed stimulation function based on said identification of a stimulation event.

9. The implantable medical device according to claim 1, wherein the processing circuitry is configured to identify, based on the sensed electrocardiogram signals, a requirement for a stimulation event and to monitor, based on the sensed electrocardiogram signals, whether the implantable pacemaker device causes a stimulation event in relation to an identification of a requirement for a stimulation event.

10. The implantable medical device according to claim 1, wherein the processing circuitry is configured to identify an anti-tachycardia stimulation event caused by the implantable pacemaker device and to cause a transmission of information concerning said anti-tachycardia stimulation event to the device external to the patient.

11. The implantable medical device according to claim 1, wherein the processing circuitry is configured to identify a requirement for an anti-bradycardia stimulation and to monitor whether the implantable pacemaker device causes an anti-bradycardia stimulation event in relation to an identification of a requirement for an anti-bradycardia stimulation.

12. The implantable medical device according to claim 1, wherein the processing circuitry is configured to trigger a data communication message to the device external to the patient based on said identification of a stimulation event, or to include said information concerning said identification of a stimulation event in a data message scheduled according to a pre-defined regular communication scheme.

13. An implantable therapy system, comprising: an implantable pacemaker device for emitting a cardiac stimulation signal for achieving an intracardiac pacing; and an implantable medical device according to claim 1.

14. The implantable therapy system according to claim 13, wherein the implantable pacemaker device is a leadless pacemaker device.

15. A method for operating an implantable medical device for providing a diagnostic and/or therapeutic cardiac function within a patient, comprising:
sensing, using a sensing arrangement of the implantable medical device electrocardiogram signals;
processing, using a processing circuitry of the implantable medical device, sensed electrocardiogram signals, wherein the processing includes: identifying, based on the sensed electrocardiogram signals, a stimulation event caused by an implantable pacemaker device and causing a transmission, using a communication circuitry of the implantable medical device, of information concerning an identification of a stimulation event to a device external to the patient.
